(19) United States
(12) Patent Application Publication
(10) Pub. No.: US 2020/0207057 A1
(43) Pub. Date: Jul. 2, 2020

(54) INTEGRATED ACTUATOR FOR EXTENDED FUNCTIONAL FABRIC

(71) Applicant: Flex Ltd., Singapore (SG)

(72) Inventors: Allison E. Bowles, Boston, MA (US); Julian Reis, Boston, MA (US); Benjamin Cooper, Hingham, MA (US); Yolita C. Nugent, Chestnut Hill, MA (US)

(21) Appl. No.: 16/728,381

(22) Filed: Dec. 27, 2019

Related U.S. Application Data
(60) Provisional application No. 62/785,894, filed on Dec. 28, 2018.

(51) Int. Cl. B32B 5/26; D04B 1/16; B32B 5/02; B32B 5/06; B32B 5/08; D04B 21/00; A61B 5/0408; A61B 5/0478; A61B 5/0492

(52) U.S. Cl. CPC B32B 5/26; B32B 2311/30; B32B 5/026; B32B 5/06; B32B 5/08; D04B 21/00; A61B 5/0408; A61B 5/0478; A61B 5/0492; D10B 2331/02; D10B 2331/04; D10B 2201/02; B32B 2250/03; B32B 2250/40; B32B 2255/02; B32B 2262/0261; B32B 2262/0276; B32B 2262/062; B32B 2255/205; B32B 2307/202; B32B 2307/204; B32B 2535/00; B32B 2457/00; B32B 2311/12; B32B 2311/08; D04B 1/16

(57) ABSTRACT

Embodiments of the disclosure provide systems and methods for producing and using a knitted fabric including one or more integrated sensors. According to one embodiment, a soft touch sensor can comprise a first knitted conductive layer of metallic-coated, plaited yarn and a second knitted conductive layer of metallic-coated, plaited yarn disposed parallel to the first knitted conductive layer. A knitted dielectric layer disposed between the first knitted conductive layer and the second knitted conductive layer. The knitted dielectric layer can be plaited into the first knitted conductive layer and the second knitted conductive layer and can form a spacer between the first knitted conductive layer and the second knitted conductive layer.

### CROSS REFERENCE TO RELATED APPLICATION

The present application claims the benefits of and priority, under 35 U.S.C. § 119(e), to U.S. Provisional Application No. 62/785,894 filed Dec. 28, 2018 by Bowles et al and entitled “Integrated Actuator for Extended Functional Fabric” of which the entire disclosure is incorporated herein by reference for all purposes.

### FIELD OF THE DISCLOSURE

Embodiments of the present disclosure relate generally to methods and systems for producing and using a knitted fabric and more particularly to producing and using a knitted fabric including one or more integrated sensors.

### BACKGROUND

Various types of sensors can be attached to or integrated into fabrics that can formed into garments or wearable accessories. For example, various types of medical sensors are commonly attached to garments, wristbands, headbands, etc., for monitoring vital signs and/or other biometrics. Monitoring using such sensors is also common in other fields such as sports and athletics. Currently, such sensors are rigid and/or not breathable which makes them uncomfortable for the wearer. Hence, there is a need for improved methods and systems for integrating sensors into fabrics.

### BRIEF SUMMARY

Embodiments of the disclosure provide systems and methods for producing and using a knitted fabric including one or more integrated sensors. According to one embodiment, a soft touch sensor can comprise a first knitted conductive layer of metallic-coated, plaited yarn and a second knitted conductive layer of metallic-coated, plaited yarn disposed parallel to the first knitted conductive layer. A metallic coating of the first conductive layer and/or the second conductive layer can comprise silver, copper, stainless steel, or other conductive metal.

A knitted dielectric layer can be disposed between the first knitted conductive layer and the second knitted conductive layer. The knitted dielectric layer can be plaited into the first knitted conductive layer and the second knitted conductive layer and can form a spacer between the first knitted conductive layer and the second knitted conductive layer. The knitted dielectric layer can comprise, for example, a plaited nylon yarn, a plaited polyester yard, or a plaited cotton yarn. The knitted dielectric layer can be integrated into a fabric sheet.

According to another embodiment, a fabric can comprise a knitted base fabric layer and one or more sensors integrated into the knitted base fabric layer. Each sensor of the one or more sensors can comprise a first knitted conductive layer of metallic-coated, plaited yarn disposed on a first surface of the knitted base fabric layer and a second knitted conductive layer of metallic-coated, plaited yarn disposed on a second surface of the knitted base fabric layer opposite the first knitted conductive layer of metallic-coated, plaited yarn. The first knitted conductive layer of metallic-coated, plaited yarn and the second knitted conductive layer of metallic-coated, plaited yarn can be plaited into the knitted base fabric layer and the based fabric layer can comprise a dielectric spacer between the first knitted conductive layer of metallic-coated, plaited yarn and the second knitted conductive layer of metallic-coated, plaited yarn. The one or more sensors can comprise one or more of a capacitive sensor, a resistive sensor, an electrocardiogram (ECG) sensor, an electroencephalogram (EEG) sensor, an electromyography (EMG) sensor, etc.

According to yet another embodiment, a method for producing a sensor-integrated fabric can comprise knitting a base fabric layer, knitting a first knitted conductive layer of metallic-coated, plaited yarn on a first surface of the knitted base fabric layer, and knitting a second knitted conductive layer of metallic-coated, plaited yarn disposed on a second surface of the knitted base fabric layer opposite the first knitted conductive layer of metallic-coated, plaited yarn. Knitting the base fabric layer and knitting the first knitted conductive layer of metallic-coated, plaited yarn can comprise plaiting the first knitted conductive layer of metallic-coated with the base fabric layer. Additionally, or alternatively, knitting the base fabric layer and knitting the second knitted conductive layer of metallic-coated, plaited yarn can comprise plaiting the second knitted conductive layer of metallic-coated with the base fabric layer.

The base fabric layer can comprise a dielectric spacer between the first knitted conductive layer of metallic-coated, plaited yarn and the second knitted conductive layer of metallic-coated, plaited yarn. For example, the base fabric layer can comprise plaited nylon yarn, a plaited polyester yard, or a plaited cotton yarn. A metallic coating of the first knitted conductive layer of metallic-coated, plaited yarn and the second knitted conductive layer of metallic-coated, plaited yarn can comprise silver, copper, stainless steel, or other conductive metal.

### BRIEF DESCRIPTION OF THE DRAWINGS

FIG. 1 illustrates a view of a knitted fabric comprising a soft sensor according to one embodiment of the present disclosure.

FIG. 2 illustrates another view of a knitted fabric comprising a soft sensor according to one embodiment of the present disclosure.

FIG. 3 illustrates yet another view of a knitted fabric comprising a plurality of soft sensors according to one embodiment of the present disclosure.

FIG. 4 is a flowchart illustrating an exemplary process for producing a sensor-integrated fabric according to one embodiment of the present disclosure.

In the appended figures, similar components and/or features may have the same reference label. Further, various components of the same type may be distinguished by following the reference label by a letter that distinguishes among the similar components. If only the first reference label is used in the specification, the description is applicable to any one of the similar components having the same first reference label irrespective of the second reference label.

### DETAILED DESCRIPTION

In the following description, for the purposes of explanation, numerous specific details are set forth in order to provide a thorough understanding of various embodiments disclosed herein. It will be apparent, however, to one skilled in the art that various embodiments of the present disclosure may be practiced without some of these specific details. The ensuing description provides exemplary embodiments only and is not intended to limit the scope or applicability of the disclosure. Furthermore, to avoid unnecessarily obscuring the present disclosure, the preceding description omits a number of known structures and devices. This omission is not to be construed as a limitation of the scopes of the claims. Rather, the ensuing description of the exemplary embodiments will provide those skilled in the art with an enabling description for implementing an exemplary embodiment. It should however be appreciated that the present disclosure may be practiced in a variety of ways beyond the specific detail set forth herein.

As used herein, the phrases “at least one,” “one or more,” “or,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” “A, B, and/or C,” and “A, B, or C” means A alone, B alone, C alone, A and B together, A and C together, B and C together, or A, B and C together.

The term “a” or “an” entity refers to one or more of that entity. As such, the terms “a” (or “an”), “one or more” and “at least one” can be used interchangeably herein. It is also to be noted that the terms “comprising,” “including,” and “having” can be used interchangeably.

It shall be understood that the term “means” as used herein shall be given its broadest possible interpretation in accordance with 35 U.S.C., Section 112, Paragraph 6. Accordingly, a claim incorporating the term “means” shall cover all structures, materials, or acts set forth herein, and all of the equivalents thereof. Further, the structures, materials or acts and the equivalents thereof shall include all those described in the summary of the disclosure, brief description of the drawings, detailed description, abstract, and claims themselves.

Various additional details of embodiments of the present disclosure will be described below with reference to the figures. While the flowcharts will be discussed and illustrated in relation to a particular sequence of events, it should be appreciated that changes, additions, and omissions to this sequence can occur without materially affecting the operation of the disclosed embodiments, configuration, and aspects.

Embodiments of the present disclosure include one or more soft touch capacitive sensors integrated into fabric by three-dimensional knitting of conductive and dielectric yarns into spacer configuration for capacitive and resistive sensors. Two conductive planes knit with silver-plated nylon yarn separated by dielectric yarn spacer (such as Nylon, Polyester, cotton and similar materials) can be integrated into knit tubular fabrics that, for example, enshroud speakers in one application. C-knit spacer can be produced on Shima Seiki SWG091N2, for example. Other machines are available to perform similar operations. Other conductive yarns can be used such as copper fiber, stainless steel and other metallic conductive materials. The conductive materials could come in a single element, plated, and coated over dielectric yarn forms. Dielectric yarns can include but are not limited to: Nylon, Polyester, cotton and similar materials. The system performance is independent of the yarn count.

Soft sensors can be integrated directly into the fabric during knitting process in a single operation, reducing the number of parts and operations in the system. Such a fabric can maintain breathability and conformability, eliminate mechanical parts without reducing functional capacity, and provide a seamless user experience. Outputs can include but are not limited to visual, audio, and mechanical actuation (light dimming sound, volume control, on/off switch, motor controller for speed, force, and/or position, system trigger), provide actuation of biometric sensing such as ECG, EEG, EMG, drug delivery, provide wound care, vital sign monitoring, etc. Industry applications can include but are not limited to audio, automotive, medical, industrial, consumer, home, wearables, aerospace, robotics, etc.

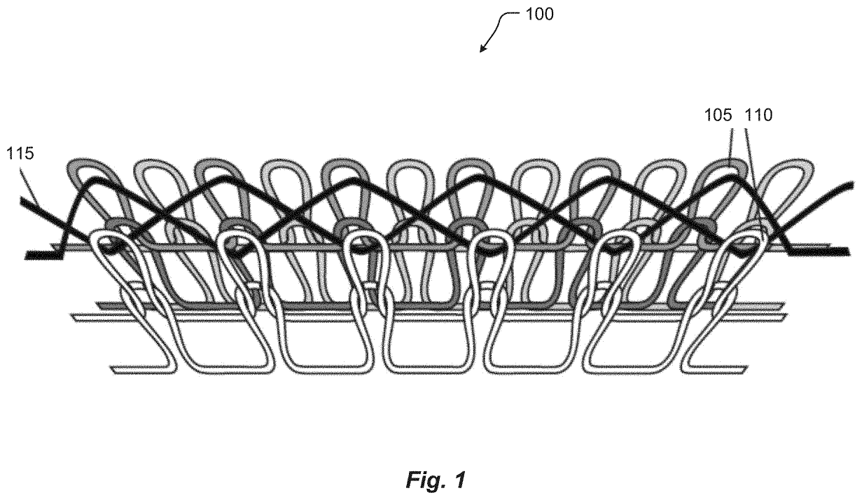

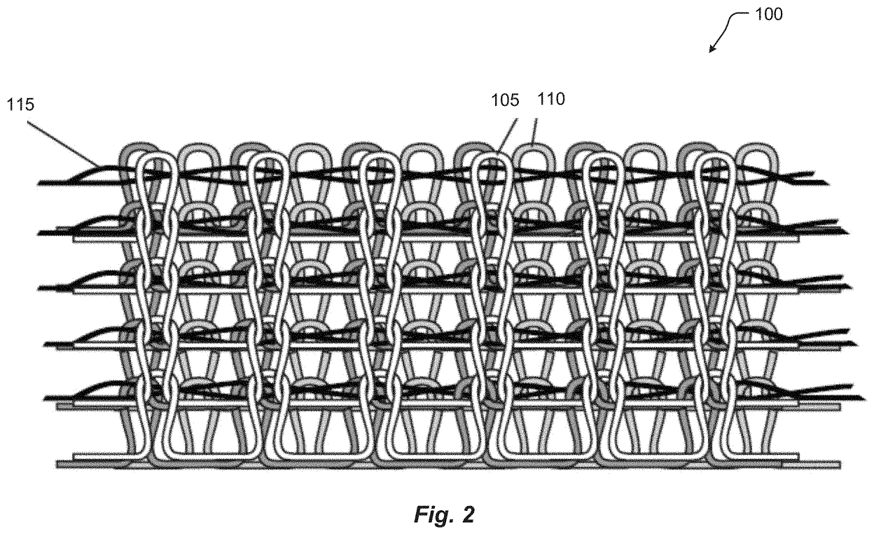

FIG. 1 illustrates a view of a knitted fabric comprising a soft sensor according to one embodiment of the present disclosure. FIG. 2 illustrates another view of a knitted fabric comprising a soft sensor according to one embodiment of the present disclosure. As illustrated in these examples, a soft touch sensor 100 can comprise a first knitted conductive layer 105 of metallic-coated, plaited yarn and a second knitted conductive layer 110 of metallic-coated, plaited yarn disposed parallel to the first knitted conductive layer 105. A metallic coating of the first conductive layer and/or the second conductive layer can comprise silver, copper, stainless steel, or other conductive metal.

A knitted dielectric layer 115 can be disposed between the first knitted conductive layer 105 and the second knitted conductive layer 110. The knitted dielectric layer 115 can be plaited into the first knitted conductive layer 105 and the second knitted conductive layer 110 and can form a spacer between the first knitted conductive layer 105 and the second knitted conductive layer 110. The knitted dielectric layer 115 can comprise, for example, a plaited nylon yarn, a plaited polyester yard, or a plaited cotton yarn. The knitted dielectric layer 115 can be integrated into a fabric sheet.

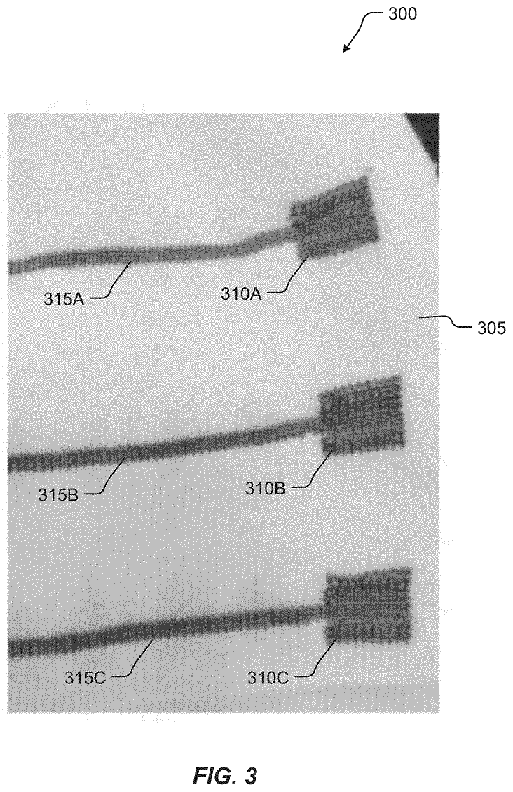

FIG. 3 illustrates yet another view of a knitted fabric comprising a plurality of soft sensors according to one embodiment of the present disclosure. As illustrated in this example, a fabric 300 can comprise a knitted base fabric layer 305 and one or more sensors 310A-310C, such as sensors 100 described above, integrated into the knitted base fabric layer 305. As described above, each sensor 310A-310C of the one or more sensors can comprise a first knitted conductive layer 105 of metallic-coated, plaited yarn disposed on a first surface of the knitted base fabric layer 305 and a second knitted conductive layer 110 of metallic-coated, plaited yarn disposed on a second surface of the knitted base fabric layer 305 (not visible here) opposite the first knitted conductive layer 105 of metallic-coated, plaited yarn. The first knitted conductive layer 105 of metallic-coated, plaited yarn and the second knitted conductive layer 110 of metallic-coated, plaited yarn can be plaited into the knitted base fabric layer 305 and the based fabric layer can comprise a dielectric spacer between the first knitted conductive layer 105 of metallic-coated, plaited yarn and the second knitted conductive layer 1110 of metallic-coated, plaited yarn. The one or more sensors can comprise one or more of a capacitive sensor, a resistive sensor, an electrocardiogram (ECG) sensor, an electroencephalogram (EEG) sensor, an electromyography (EMG) sensor, etc.

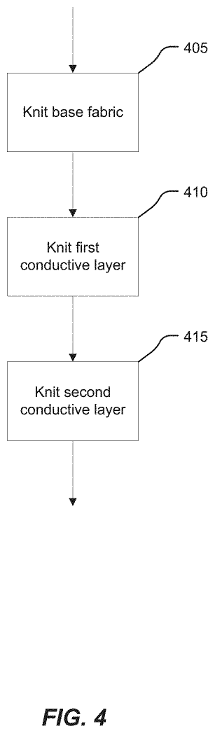

FIG. 4 is a flowchart illustrating an exemplary process for producing a sensor-integrated fabric according to one embodiment of the present disclosure. As illustrated in this example, producing a sensor-integrated fabric can comprise knitting 405 a base fabric layer 305, knitting 410 a first knitted conductive layer 105 of metallic-coated, plaited yarn on a first surface of the knitted base fabric layer 305, and knitting 415 a second knitted conductive layer 110 of metallic-coated, plaited yarn disposed on a second surface of the knitted base fabric layer 305 opposite the first knitted conductive layer 105 of metallic-coated, plaited yarn. Knitting 405 the base fabric layer 305 and knitting 410 the first knitted conductive layer 105 of metallic-coated, plaited yarn can comprise plaiting the first knitted conductive layer 105 of metallic-coated with the base fabric layer 305. Additionally, or alternatively, knitting 405 the base fabric layer 305 and knitting 415 the second knitted conductive layer 110 of metallic-coated, plaited yarn can comprise plaiting the second knitted conductive layer 110 of metallic-coated with the base fabric layer 305.

The base fabric layer 305 can comprise a dielectric layer 115 or spacer between the first knitted conductive layer 105 of metallic-coated, plaited yarn and the second knitted conductive layer 110 of metallic-coated, plaited yarn. For example, the base fabric layer 305 can comprise plaited nylon yarn, a plaited polyester yard, or a plaited cotton yarn. A metallic coating of the first knitted conductive layer 105 of metallic-coated, plaited yarn and the second knitted conductive layer 110 of metallic-coated, plaited yarn can comprise silver, copper, stainless steel, or other conductive metal.

The present disclosure, in various aspects, embodiments, and/or configurations, includes components, methods, processes, systems, and/or apparatus substantially as depicted and described herein, including various aspects, embodiments, configurations embodiments, sub-combinations, and/or subsets thereof. Those of skill in the art will understand how to make and use the disclosed aspects, embodiments, and/or configurations after understanding the present disclosure. The present disclosure, in various aspects, embodiments, and/or configurations, includes providing devices and processes in the absence of items not depicted and/or described herein or in various aspects, embodiments, and/or configurations hereof, including in the absence of such items as may have been used in previous devices or processes, e.g., for improving performance, achieving ease and\or reducing cost of implementation.

The foregoing discussion has been presented for purposes of illustration and description. The foregoing is not intended to limit the disclosure to the form or forms disclosed herein. In the foregoing Detailed Description for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the description has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A soft touch sensor comprising:
a first knitted conductive layer of metallic-coated, plaited yarn;
a second knitted conductive layer of metallic-coated, plaited yarn disposed parallel to the first knitted conductive layer; and
a knitted dielectric layer disposed between the first knitted conductive layer and the second knitted conductive layer, the knitted dielectric layer plaited into the first knitted conductive layer and the second knitted conductive layer and forming a spacer between the first knitted conductive layer and the second knitted conductive layer.

2. The soft touch sensor of claim 1, wherein the knitted dielectric layer is integrated into a fabric sheet.

3. The soft touch sensor of claim 1, wherein the knitted dielectric layer comprises a plaited nylon yarn.

4. The soft touch sensor of claim 1, wherein the knitted dielectric layer comprises a plaited polyester yarn.

5. The soft touch sensor of claim 1, wherein the knitted dielectric layer comprises a plaited cotton yarn.

6. The soft touch sensor of claim 1, wherein a metallic coating of the first conductive layer or second conductive layer comprises silver.

7. The soft touch sensor of claim 1, wherein a metallic coating of the first conductive layer or second conductive layer comprises copper.

8. The soft touch sensor of claim 1, wherein a metallic coating of the first conductive layer or second conductive layer comprises stainless steel.

9. A fabric comprising:
a knitted base fabric layer;
one or more sensors integrated into the knitted base fabric layer, each sensor of the one or more sensors comprising a first knitted conductive layer of metallic-coated, plaited yarn disposed on a first surface of the knitted base fabric layer and a second knitted conductive layer of metallic-coated, plaited yarn disposed on a second surface of the knitted base fabric layer opposite the first knitted conductive layer of metallic-coated, plaited yarn, wherein the first knitted conductive layer of metallic-coated, plaited yarn and the second knitted conductive layer of metallic-coated, plaited yarn are plaited into the knitted base fabric layer and the based fabric layer comprises a dielectric spacer between the first knitted conductive layer of metallic-coated, plaited yarn and the second knitted conductive layer of metallic-coated, plaited yarn.

10. The fabric of claim 9, wherein at least one of the one or more sensors comprises a capacitive sensor.

11. The fabric of claim 9, wherein at least one of the one or more sensors comprises a resistive sensor.

12. The fabric of claim 9, wherein at least one of the one or more sensors comprises an electrocardiogram (ECG) sensor.

13. The fabric of claim 9, wherein at least one of the one or more sensors comprises an electroencephalogram (EEG) sensor.

14. The fabric of claim 9, wherein at least one of the one or more sensors comprises an electromyography (EMG) sensor.

15. A method for producing a sensor-integrated fabric, the method comprising
knitting a base fabric layer;
knitting a first knitted conductive layer of metallic-coated, plaited yarn on a first surface of the knitted base fabric layer; and
knitting a second knitted conductive layer of metallic-coated, plaited yarn disposed on a second surface of the knitted base fabric layer opposite the first knitted conductive layer of metallic-coated, plaited yarn.

16. The method of claim 15, wherein knitting the base fabric layer and knitting the first knitted conductive layer of metallic-coated, plaited yarn comprises plaiting the first knitted conductive layer of metallic-coated with the base fabric layer.

17. The method of claim 16, wherein knitting the base fabric layer and knitting the second knitted conductive layer of metallic-coated, plaited yarn comprises plaiting the second knitted conductive layer of metallic-coated with the base fabric layer.

18. The method of claim 17, wherein the based fabric layer comprises a dielectric spacer between the first knitted conductive layer of metallic-coated, plaited yarn and the second knitted conductive layer of metallic-coated, plaited yarn.

19. The method of claim 18, wherein the base fabric layer comprises plaited nylon yarn, a plaited polyester yard, or a plaited cotton yarn.

20. The method of claim 19, wherein a metallic coating of the first knitted conductive layer of metallic-coated, plaited yarn and the second knitted conductive layer of metallic-coated, plaited yarn comprises silver, copper, or stainless steel.
